(19) United States
(12) Patent Application Publication
(10) Pub. No.: US 2017/0246271 A1
(43) Pub. Date: Aug. 31, 2017

(54) INNATE IMMUNE MODULATING MOLECULE COMPLEX FOR THE ACTIVATION OF THE INNATE IMMUNE SYSTEM OF AQUATIC ANIMALS AND METHODS THEREFOR

(71) Applicant: Perkins Biosystems, LLC, Manhattan, KS (US)

(72) Inventors: Timothy L. Miller, Lincoln, NE (US); Gary L. Faltys, Oakland, NE (US)

(21) Appl. No.: 15/509,634

(22) PCT Filed: Sep. 8, 2015
(86) PCT No.: PCT/US15/48980

§ 371 Date: Mar. 8, 2017

Related U.S. Application Data
(60) Provisional application No. 62/047,380, filed on Sep. 8, 2014.

(51) Int. Cl. A61K 39/00; A61K 39/39

(52) U.S. Cl. CPC A61K 39/0005; A61K 39/39; A61K 2039/53; A61K 2039/552; A61K 2039/55555

(57) ABSTRACT

A method for application of an innate immune modulating molecule complex to activate the innate immune system of aquatic animals includes selecting a recombinant DNA with characteristics configured for activating aquatic animal macrophages, such as by inducing an innate immune response in the animal. The method for application of an innate immune modulating molecule complex also includes selecting an adjuvant with characteristics favorable for activating aquatic animal macrophages and causing the release of cytokines. The stimulant recombinant DNA is combined with the adjuvant to form an innate immune modulating molecule (I2M2 complex). The I2M2 complex induces a synergistic response in macrophages that causes activation and proliferation of macrophages, expulsion of or inhibition of pathogens from the macrophages, and release of cytokines that aid in normal innate and acquired immune responses of fish.

### BACKGROUND

Infectious pathogens are considered to be one of the largest threats to aquaculture and the productivity of the fish farming industry. Rapid growth and intensification in the aquaculture industry have increased the prevalence and complexity of health threats. The high density of animals used in large-scale commercial fish farming leads to the rapid spread of pathogens. Some infectious pathogens can travel rapidly from fish to fish either by direct contact or by indirect contact via water, vectors, or equipment. Wild fish can act as carriers, transmitting infectious pathogens (e.g., viruses or bacteria) to farmed fish. Conversely, infectious pathogens can develop in the condensed populations of farmed fish, which can be transmitted to wild fish. Of particular concern are infectious pathogens that could develop in condensed fish populations that kill rather than weaken the host, particularly where the pathogens could be transmitted to wild fish populations. Vaccines offer a targeted approach for prevention of many infectious pathogens and play a key role in controlling infectious diseases in fish.

### SUMMARY

An innate immune modulating molecule complex, methods for application of an innate immune modulating molecule complex to activate the innate immune system of aquatic animals, and methods for deriving the innate immune modulating complex are provided. The innate immune modulating molecule complex includes a supercoiled covalently closed recombinant plasmid DNA, where the supercoiled covalently closed recombinant plasmid DNA comprises characteristics configured for inducing an innate immune response in aquatic animals. The innate immune modulating molecule complex also includes an adjuvant complexed with the supercoiled covalently closed recombinant plasmid DNA, where the adjuvant comprises characteristics configured for inducing cytokine release from macrophages in aquatic animals. The method for application of an innate immune modulating molecule complex includes selecting a supercoiled covalently closed recombinant plasmid DNA, where the supercoiled covalently closed recombinant plasmid DNA comprises characteristics configured for inducing an innate immune response in aquatic animals. The method also includes selecting an adjuvant, where the adjuvant comprises characteristics configured for inducing cytokine release from macrophages in aquatic animals. The recombinant DNA is combined with the adjuvant to form a stable innate immune modulating molecule complex.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### DRAWINGS

The Detailed Description is described with reference to the accompanying figures. The use of the same reference numbers in different instances in the description and the figures may indicate similar or identical items.

FIG. 1A is a flow diagram illustrating an example method for application of an innate immune modulating molecule complex to activate the innate immune system of aquatic animals in accordance with example implementations of the present disclosure.

FIG. 1B is a diagrammatic illustration of a plasmid DNA incorporated as a portion of an innate immune modulating molecule complex in accordance with example implementations of the present disclosure.

FIG. 2 is a TNF-α (tumor necrosis factor alpha) stimulation assay for a plurality of adjuvant formulations in accordance with example implementations of the present disclosure.

FIG. 3 is a table showing absorbance values measuring BudR (bromodeoxyuridine) incorporation into dsDNA (double stranded DNA) indicating ASK (Atlantic Salmon kidney/head cells) cell proliferation in accordance with example implementations of the present disclosure.

FIG. 4 is a chart showing BudR incorporation to measure ASK cell proliferation in accordance with example implementations of the present disclosure.

FIG. 5 is a table of C(t) (cycle threshold) values on ASK Cells infected with P. salmonis and treated following infection in accordance with example implementations of the present disclosure.

FIG. 6 is a chart of TNF-α expression by individual samples in accordance with example implementations of the present disclosure.

FIG. 7 is a bar graph of TNFα expression by individual samples in accordance with example implementations of the present disclosure.

FIG. 8 is a randomized analysis of variance and Tukey's range test dose determination study in accordance with example implementations of the present disclosure.

FIG. 9 is a chart of IL-12 (Interleukin 12 cytokine) expression by individual samples in accordance with example implementations of the present disclosure.

FIG. 10 is a bar graph of IL-12 expression grouped by hour in accordance with example implementations of the present disclosure.

FIG. 11 is a box-and-whisker plot of serum concentration of TNFα in accordance with example implementations of the present disclosure.

FIG. 12 is a box-and-whisker plot of serum concentration of IL-12 in accordance with example implementations of the present disclosure.

FIG. 13 is a chart of TNFα expression by sample date resulting from a Salmon Rickettsia Syndrome (SRS) challenge study.

FIG. 14 is a chart of TNFα expression by treatment group resulting from an SRS challenge study.

FIG. 15 is a chart of TNFα expression by treatment group and sample date resulting from an SRS challenge study.

### DETAILED DESCRIPTION

Overview

Infectious pathogens are one of the most significant threats to the fish farming industry, leading to high morbidity and mortality rates, and decreased productivity. The salmon industry is plagued by Salmon Rickettsia Syndrome (SRS) and Infectious Salmon Anemia Virus (ISAV). Vaccines play an important role in the control of these infectious diseases. Traditional vaccines developed for SRS and ISAV have been targeted at acquired immunity, that is, immune responses developed from historical interactions with foreign substances (e.g., antigens). These vaccines have had limited success in fish because of acquired immune systems that appear to have limitations in responses to vaccine usage, as evidenced by cell populations that are limited or lacking in fish, such as mature memory cells, in comparison to other animals. One way to overcome the challenges presented by a less mature acquired immune system is to target innate immunity rather than acquired immunity. Nucleic acids (e.g., DNA, RNA, and so forth) are thought to induce innate-immune responses in other mammals such as bovines. Portions of DNA can induce endosomal innate responses by attaching to Toll receptors on endosomal organelles in macrophages and stimulating the macrophages. Portions of DNA can also induce cytosolic innate signaling to modulate the immune response in cells. The infectious pathogens representing SRS and ISAV invade endosomal organelles of macrophages using the host cell to replicate the pathogens and inactivate the host cell. One challenge of developing fish vaccines is to develop methods of using recombinant DNA technology to target innate immunity, such as administering exogenous DNA as a preventative or therapy in disease challenged macrophage cells to modulate the formally inactive cells.

Accordingly, an innate immune modulating molecule complex (e.g., I2M2 complex described herein) and method for application of an innate immune modulating molecule complex to activate the innate immune system of aquatic animals is described herein. A stimulant recombinant DNA is selected with characteristics favorable for inducing an innate immune response in aquatic animals (e.g., fish, shrimp, and so forth). The inducement of an innate immune response can include initiating innate signaling systems of cells of the immune system, both lymphoid and myeloid in nature. For example, macrophages are cells of the myeloid lineage and are also a target of invading pathogens, such as Pisciricketsia salmonis, whereby the pathogen replicates and accumulates in the macrophage and bypasses or blocks the normal immunoresponsive features of the macrophage. The stimulant recombinant DNA can facilitate macrophage proliferation and/or cytokine expression. Further, an adjuvant is selected with characteristics favorable for inducing cytokine release from macrophages in aquatic animals. The stimulant recombinant DNA is combined with the adjuvant to form an innate immune modulating molecule complex (i.e., the I2M2 complex). The I2M2 complex induces a synergistic response in macrophages that causes activation and proliferation of macrophages, expulsion of or inhibition of pathogens from the macrophages, and release of cytokines that aid in normal innate and acquired immune responses of fish.

Example Implementations

An innate immune modulating molecule complex (e.g., I2M2 complex) in accordance with an example implementation of the present disclosure includes a plasmid DNA stimulant and an adjuvant. In implementations, a plasmid DNA stimulant with characteristics favorable for activating the macrophages of aquatic animals and for inducing an innate immune response in the aquatic animals is selected. In implementations, the plasmid DNA stimulant includes supercoiled circular covalently closed DNA strands (CCCD) isolated as a recombinant DNA modified from bacteria plasmid. Without being limited to any specific activation pathway, the plasmid DNA stimulant binds to and/or activates both cytosolic and endosomal receptors of macrophages in order to induce an innate immune response from the cell. For example, the plasmid DNA stimulant can induce an endosomal response by binding to Toll receptors on endosomal organelles in macrophages and stimulating the macrophages.

The innate immune modulating molecule complex includes an adjuvant. In implementations, an adjuvant with characteristics favorable for activating aquatic animal macrophages is selected. For example, activation of the aquatic animal macrophage can include inducing cytokine release from the macrophages that aid in normal innate and acquired immune responses of fish. In implementations, the adjuvant is ENABL 7050101 (a reagent from VaxLiant, Lincoln, Neb., Catalog No. 7050101, hereinafter referred to as “ENABL adjuvant” or “Adjuvant C”). ENABL adjuvant was screened in mouse cells and provided an ability to activate macrophages and cause cells to secrete cytokines (e.g., TNFα (tumor necrosis factor alpha)). The adjuvant may independently induce cytokine production through a non-replication dependent mechanism. The combination of the plasmid DNA and the adjuvant in the I2M2 complex can induce synergistic macrophage activation effects, induce expulsion or inhibition of infectious pathogens from macrophages, and enhance cell proliferation. Example inhibition mechanisms include walling off a pathogen to render the pathogen cryptic, dissolving the pathogen, processing the pathogen for class I or class II immune presentation, and so forth.

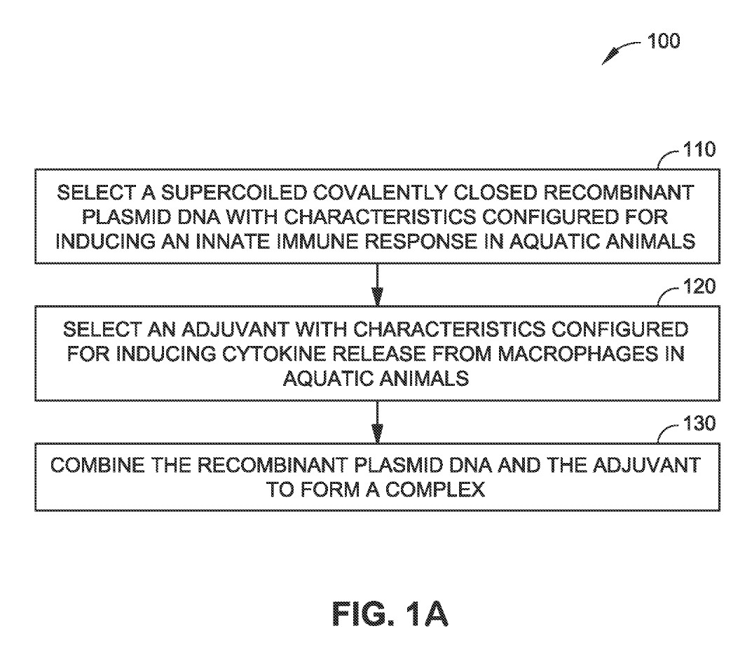

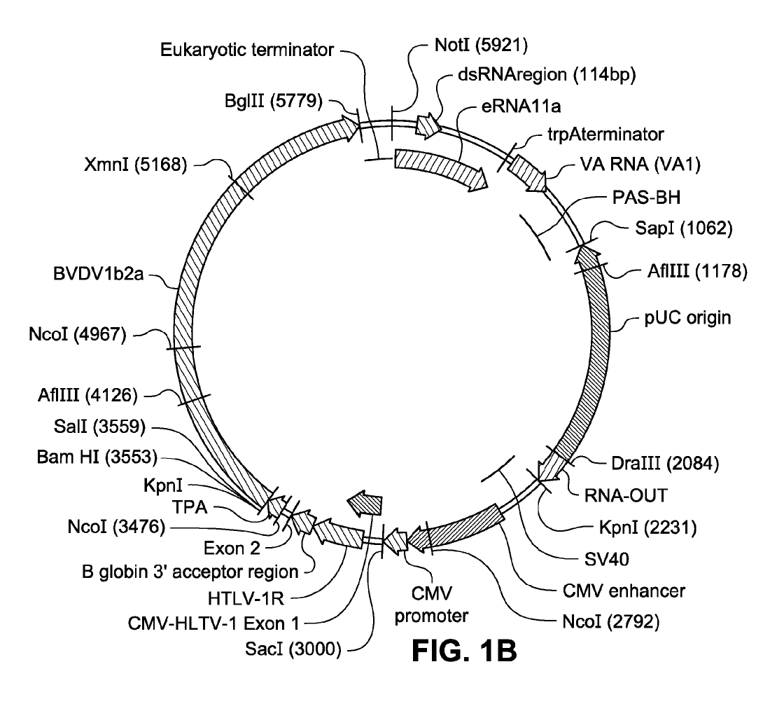

FIG. 1A illustrates a method 100 in accordance with an example implementation of the present disclosure. A plasmid DNA stimulant is selected with characteristics favorable for inducing an innate immune response in aquatic animals (e.g., activation of aquatic animal macrophages) (Block 110). In implementations, the plasmid DNA stimulant includes supercoiled circular covalently closed DNA strands (CCCD) isolated as a recombinant DNA modified from bacteria plasmid. The plasmid DNA can induce an endosomal response by binding to Toll receptors on endosomal organelles in macrophages and stimulating the macrophages. The plasmid DNA can also induce cytosolic innate signaling to modulate the immune response. Induction of the innate immune system in mammals and vertebrates has been shown to be an important feature as one of the primary response mechanisms against infectious disease agents. In mammals the innate immune response is also integral to the acquired immune response. A number of biologically derived materials have been shown to induce the innate response including bacterial cell walls, yeast cell walls, flagellum, dsRNA, and DNA. Nucleic acids that are exposed to the cell either from cell damage, infection, or incomplete clearance of inflammation can evoke strong immune responses (see, e.g., Takaoka, A. et. al. 2007, DAI (DLM-1ZBP1) is a cytosolic DNA sensor and an activator of innate immune response, Nature: 228: 501-506, which is hereby incorporated by reference in its entirety). More recent evidence indicates that DNA plasmids can induce both antitumor and anti-infectious response when transfected or delivered in a liposomal-DNA complex. Furthermore, the plasmid DNA does not necessarily need to express a gene in the cell to evoke these responses (see, e.g., Li, S. et. al. 2005, Induction of IFN-Regulated Factors and Antitumoral Surveillance by Transfected Placebo Plasmid DNA, Molecular Therapy, 11: 112-119; and Gowen, B. B., et. al. 2006, Protective immunity against acute phleboviral infection elicited through immunostimulatory cationic liposome-DNA complexes, Antiviral Research, 69: 165-172, each of which is incorporated by reference in its entirety).

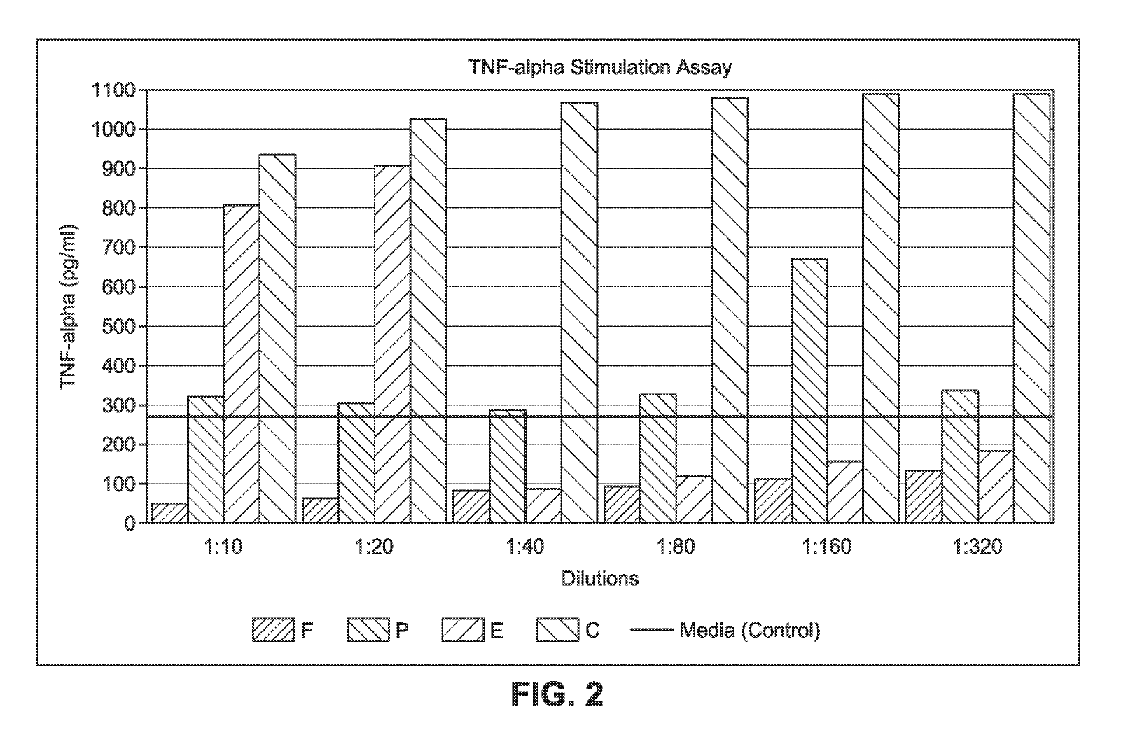

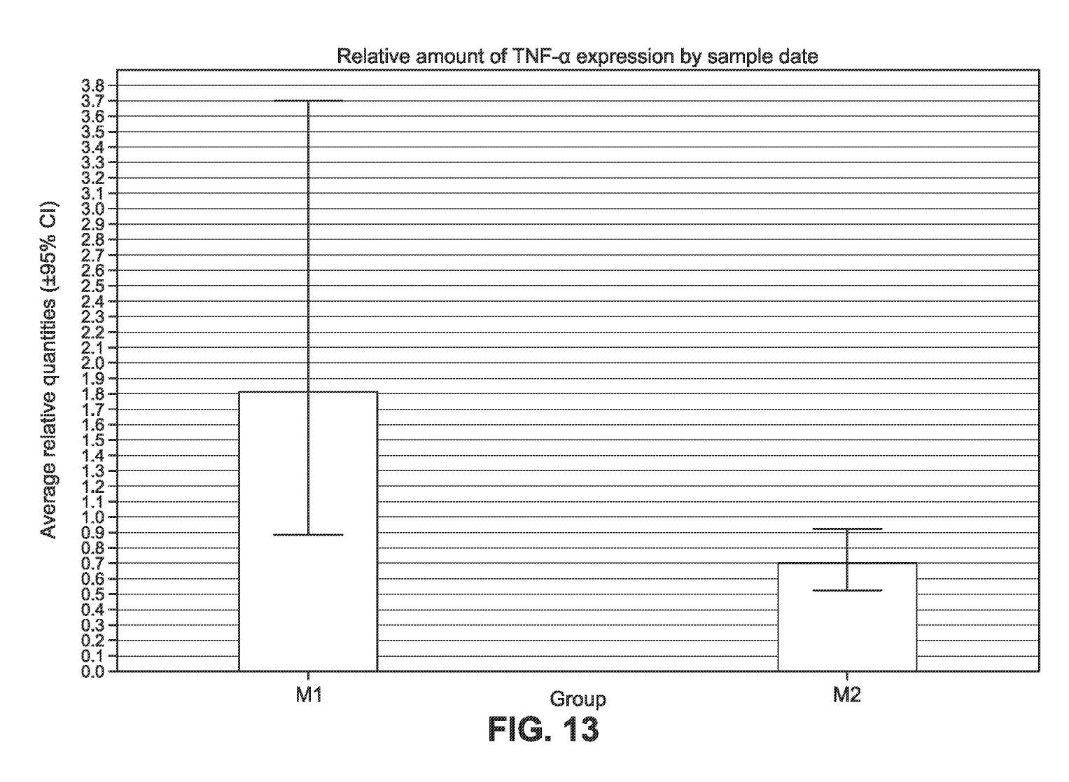

In implementations, the plasmid DNA is a double stranded covalently closed plasmid DNA. The backbone plasmid DNA sequence is derived from a core of plasmid DNA vectors all using the retinoic acid inducible gene-1 (RIG-I) activating DNA sequence. Retinoic-acid-inducible gene 1 (RIG-I) (designation-eRNA41H) is one of the cytoplasmic double stranded RNA (dsRNA) pattern receptors required for innate immune activation in response to viral infection. Nature Technology, Inc (NTC) has designed four plasmids that can express genes under normal cytoplasmic conditions (NTC8685-eRNA41H), proteosome attachment (NTC8684-eRNA41H), extracellular secretion NTC8682-eRNA41H, and endosomal secretion NTC8681-eRNA41H. This plasmid vector platform containing the eRNA41H DNA sequence combines high level RNA-mediated type I IFN activation and has high yield plasmid production in manufacturing (2.6 gm/L). Information about the NTC8682-eRNA41H plasmid, such as construction, map, and sequence information can be found, for example, in the Targeting RIG-I activating Expression Vectors Instruction Manual, version 2 (April 2013), obtained online at http://www.natx.com/assets/ntc8681-ntc8682-ntc8684-ntc8685-erna41h-user-manual-2-0.pdf, which is incorporated herein by reference in its entirety. The plasmid designated NTC8682-eRNA41H used has the sequence for bovine viral diarrhea virus (BVDV) polyprotein from subtype 1b (526 isolate) and 2a (426 isolate). The final plasmid containing the BVDV1b2a glycoprotein insert is designated NTC8682-2RNA41H-BVDV1b-2a E2T, and is shown in FIG. 1B. This DNA plasmid when combined with a lipodial complex, e.g., the ENABL adjuvant, activates ASK (Atlantic Salmon Kidney) macrophage cell line in vitro, and has shown to, inter alia, provide a substantial, synergistic increase in cytokine expression (e.g., as shown in FIGS. 2-12) which surprisingly extended to testing in live salmon (as shown in FIGS. 13-15 and described herein with reference to Example 1). This plasmid DNA contains no known fish DNA specific sequence and expresses no known gene pathogenic to fish. In implementations, the plasmid DNA comprises greater than approximately 85% supercoiled plasmid DNA (e.g., NTC8682-eRNA41H-BVDV1b-2a sourced from Nature Technology, Lincoln, Neb.).

The method of FIG. 1A also includes selecting an adjuvant with characteristics favorable for activating aquatic animal macrophages and favorable for causing the release of cytokines from macrophages Block 120). In implementations, the adjuvant is ENABL adjuvant. In implementations, the adjuvant can induce synergistic effects with the plasmid DNA and enhance cell proliferation. The adjuvant may also independently induce cytokine production through a non-replication dependent mechanism.

The method of FIG. 1A also includes combining the recombinant DNA with the adjuvant to form an innate immune modulating molecule complex (I2M2 complex; Block 130). In implementations, the I2M2 complex includes the plasmid DNA (e.g., an innate immune modulating molecule) formulated in a lipodial emulsion adjuvant. In implementations, the I2M2 complex induces both endosomal and cytosolic innate immune responses. For example, the combination of the plasmid DNA and the adjuvant in the I2M2 complex can induce synergistic macrophage activation effects, induce expulsion or inhibition of infectious pathogens from macrophages, and enhance cell proliferation. The expulsion of pathogens from the macrophages can stimulate innate immunity by inducing natural infectious pathogen resistance. Example inhibition mechanisms include walling off a pathogen to render the pathogen cryptic, dissolving the pathogen, processing the pathogen for class I or class II immune presentation, and so forth. The I2M2 complex induces a unique activation pathway that stimulates macrophage proliferation, which is not activated by either the plasmid DNA or the adjuvant alone, thereby employing a synergistic response.

FIGS. 2-12 illustrate analytical analyses indicating the success of the I2M2 complex in reducing rickettsia in vitro. In testing phase one, Salmon Head Kidney (SHK) and Atlantic Salmon Kidney cells (ASK) were selected and propagated because they are established macrophage cell lines from fish. Mouse cells (RAW) were also propagated. ASK and SHK were cultured to produce a lab stock. Adjuvants were screened in RAW. FIGS. 13-15 represent analytical analyses indicating increased cytokine expression as a result of a challenge study in Atlantic Salmon, as described with reference to Example 1 below.

FIG. 2 illustrates the activation of cytokine production of TNFα in RAW cells treated with four different adjuvant formulations. RAW cells were plated and after 24 hours of incubation, the media was removed and the different treatments diluted in cell media were added to the cells. Control cells received media alone, and treated cells received Vaxliant adjuvants F, E, C, or P. The cells were incubated with the treatments for a total of four hours. Supernatants were removed and stored at −80° C. The supernatants were thawed and aliquots were diluted and tested in duplicate for TNFα cytokine expression. Background cytokine production was considered to be about 300 ELISA absorption units (Media Control threshold line). Treatment with adjuvants E, C, and P resulted in a TNFα expression above background. The three formulations showed proliferation of TNFα using dilutions described above, however, ENABL adjuvant retained stimulation out to 1:320 dilution, whereas, treatment with adjuvants E and P resulted in increased TNFα expression at specific concentrations (1:10 and 1:20 for E and 1:160 for P). Treatment with adjuvant F resulted in no significant induction of TNFα using this method of cytokine proliferation. Results indicated an ability of ENABL adjuvant to activate macrophages and cause cells to secrete cytokine TNFα.

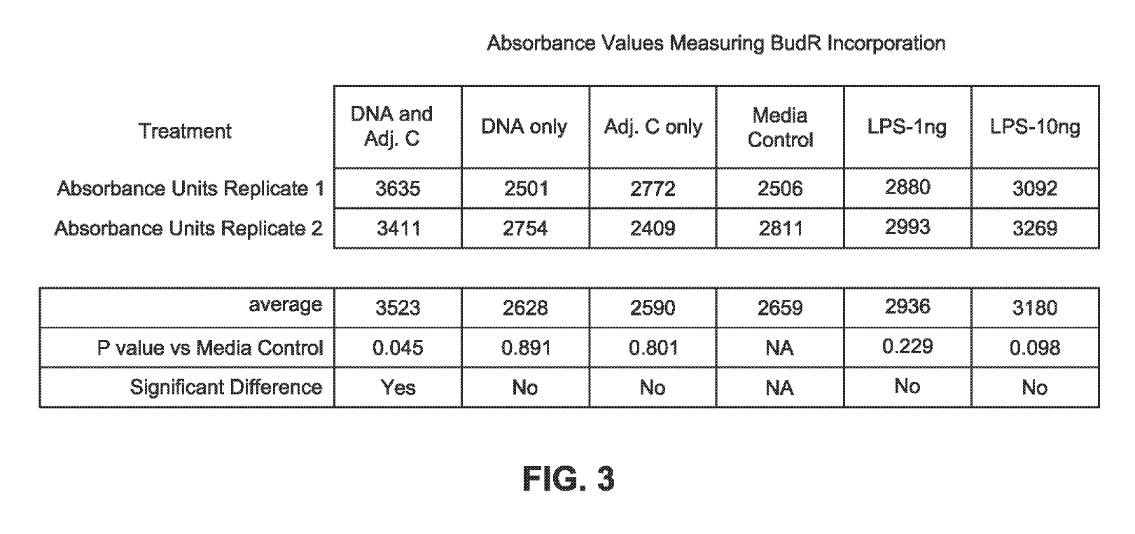

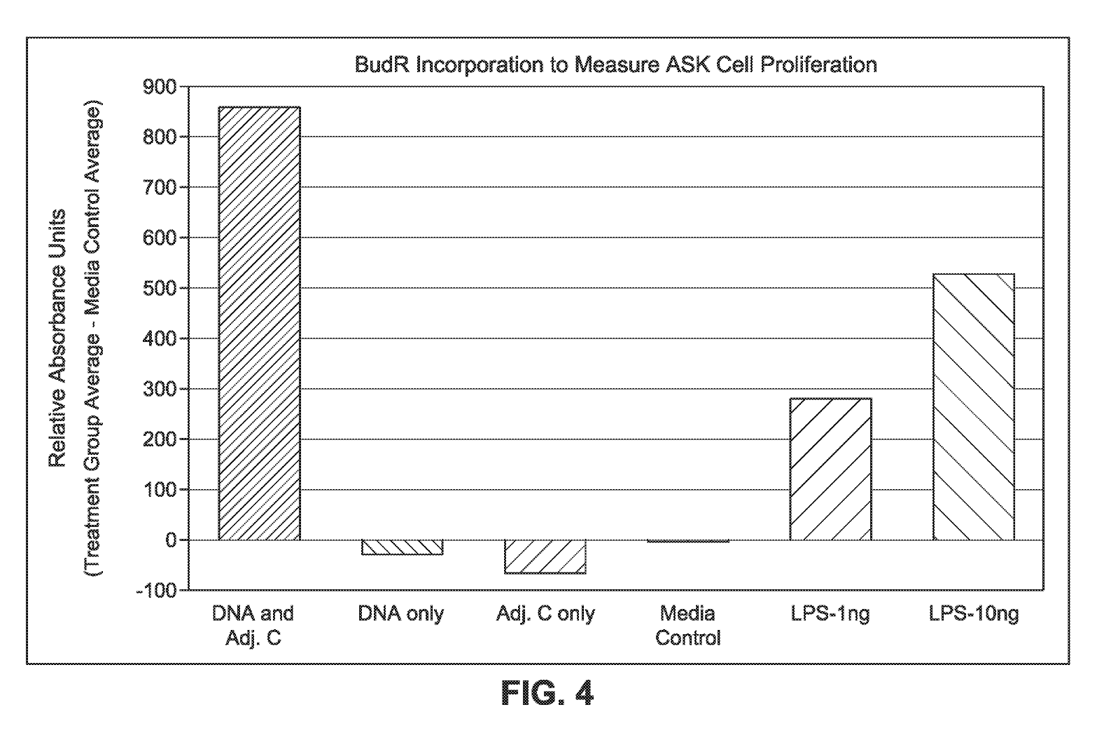

FIGS. 3 and 4 illustrate the activation of the fish ASK macrophage cell line measured by the incorporation of BudR (bromodeoxyuridine) into newly replicated genomic dsDNA (double stranded DNA). ASK cells were plated and allowed to incubate for 3-4 days or 90% confluency. Spent media was replaced with one mL of media containing the following treatments: 1 ng lipopolysaccharide (LPS), 10 ng LPS, 100 ng plasmid DNA alone, 1:500 dilution of ENABL adjuvant alone, Media alone, and 100 ng plasmid DNA complexed with 1:500 dilution of ENABL adjuvant, and allowed to incubate for 24 hours in media containing BudR. The two concentrations of LPS were used as positive controls. After the 24 hour incubation, monolayers were fixed and BudR incorporation was detected using a monoclonal antibody in an ELISA format. The treatment with DNA or adjuvant alone did not show significant proliferation compared to the negative media-only control. LPS at 1 ng and 10 ng showed a dose response with a 10% and 19% increase in proliferation, respectively; although these increases were not considered significant. In contrast, the treatment group containing the DNA (1 ng) complexed with ENABL adjuvant (1:500 dilution of 5X stock) provided a proliferation of 33% and was significant (P<0.05). The combination of Plasmid DNA and ENABL adjuvant caused proliferation of ASK cell DNA. Such proliferation can be significant due to the slow growth rate of the cells, particularly where the cells have reduced cell cycle reproduction periods.

These data show that the I2M2 complex activates macrophages in vitro and can potentially activate or prime macrophages of salmon in vivo. The data also suggests that ENABL adjuvant alone may induce cytokine production through a non-replication dependent mechanism. Without being bound to any particular biological activation pathway, it is believed that when ENABL adjuvant is complexed with plasmid DNA, a different activation pathway is induced which stimulates proliferation.

In testing phase two, the effect of the I2M2 on the presence of rickettsia DNA in cells was evaluated. A polymerase chain reaction (PCR) assay was developed to detect the presences of P. salmonis DNA. ASK cells were infected with P. salmonis (rickettsia) at dilutions of 10⁻¹ to 10⁻⁵ during a four hour incubation period. The 10⁻¹ dilution represents 10,000 rickettsia per cell, while 10⁻⁵ dilution represents 1 rickettsia per cell. The cells were treated with I2M2 before (Preventative) and after (Therapeutic) the challenge with rickettsia. PCR gel templates were used to evaluate infection with rickettsia and the response to I2M2. This is a qualitative test that illustrates whether rickettsia DNA is or is not present in a cell. The well of the PCR gel template indicates rickettsia DNA by florescence, such that a rickettsia infection fluoresces. During analysis, non-infected wells did not exhibit florescence and therefore verifies that the PCR method is specific for P. salmonis. The results of the PCR assay indicate a reduction in signal (amount of florescence) between uninfected wells and I2M2/adjuvant wells for treatment following infection, indicating that I2M2 may be a good therapeutic agent.

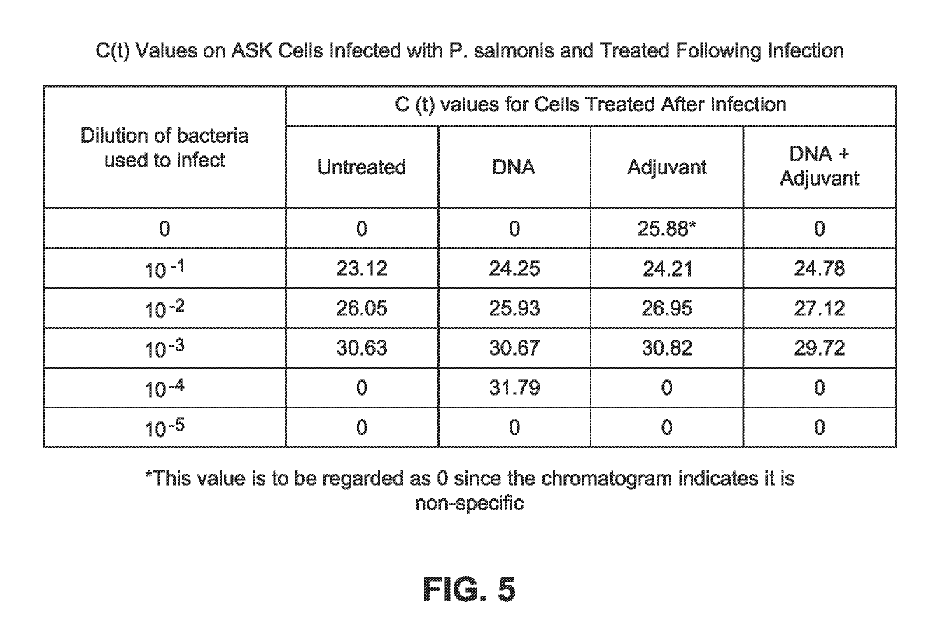

FIG. 5 illustrates quantitative real time PCR results by gel electrophoresis. Results are reported as C(t) (cycle threshold) values. When reporting data with C(t) values, a lower number comparatively indicates that more template DNA is present than in a sample with a higher C(t) value. Results may suggest that the I2M2 complex reduced the rickettsia for treatment following infection. For example: The 10⁻¹ dilution showed a C(t) value of 23.12 for untreated and a C(t) value of 24.78 for I2M2 complex.

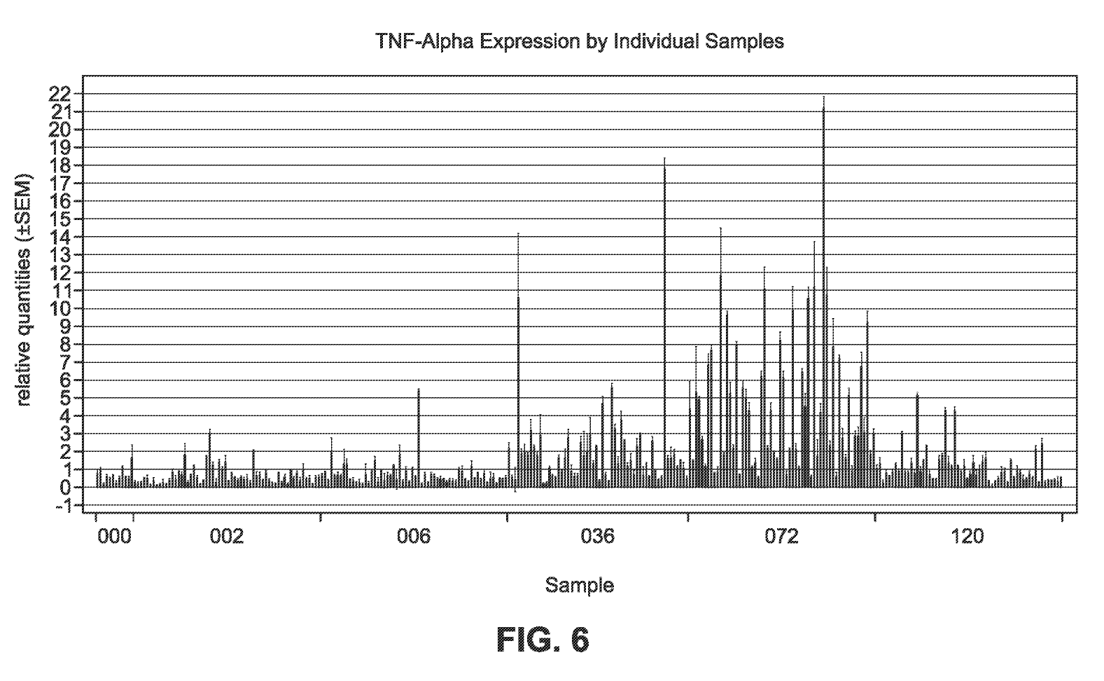

FIGS. 6-10 illustrate the results of a dose determination study conducted to characterize the effect of a single intraperitoneal injection of an innate immune modulation molecule (I2M2) on TNFα gene expression, Interleukin-12 (IL-12) gene expression, and cytokine concentrations in serum of salmon. Three hundred fish were treated in five treatment groups. The doses of I2M2 administered were 0, 1, 5, 10, or 20 mcg per fish. Blood and head kidney were collected at 0, 2, 6, 36, 72, and 120 hours following I2M2 administration. No adverse events were reported at any dose or time period.

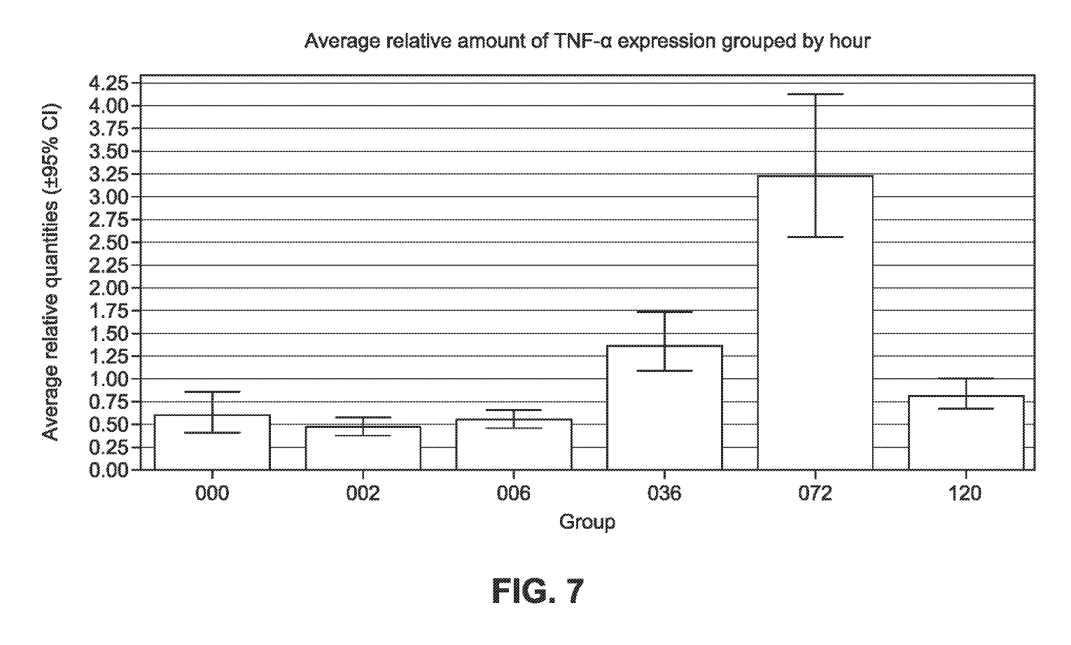

FIGS. 6 and 7 show relative amount of TNFα gene expression. A significant increase of TNFα gene expression was found in the samples labeled “72 hours” with respect to basal samples (0 hours).

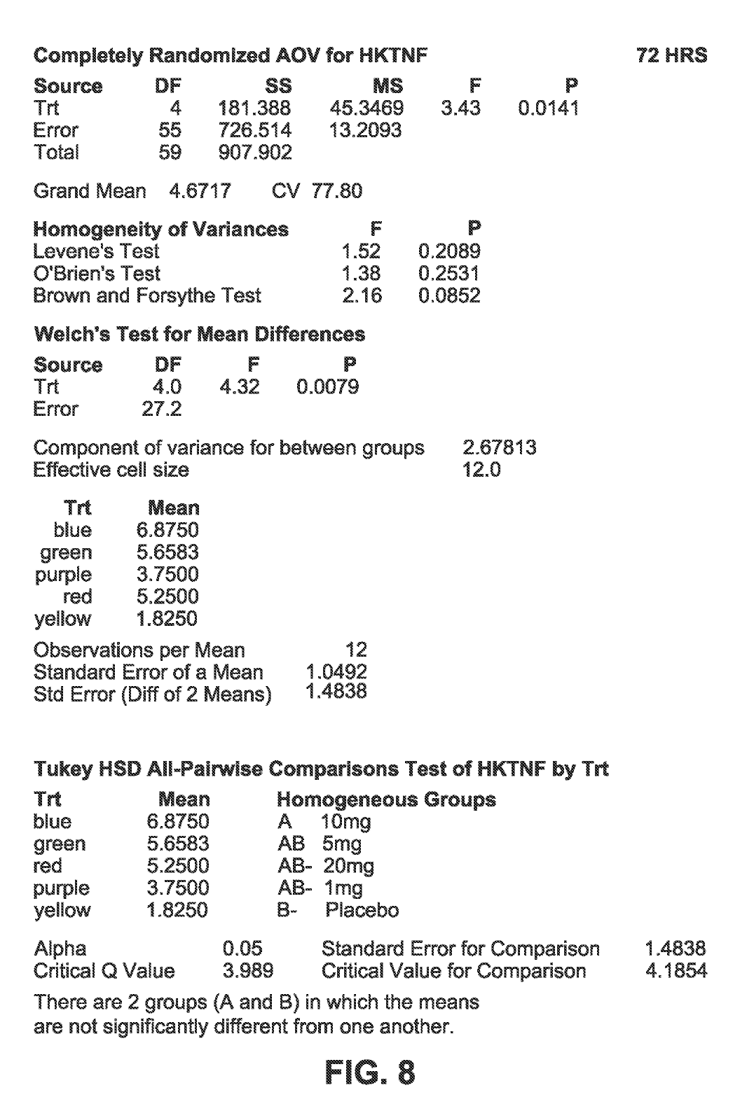

FIG. 8 shows a randomized analysis of variance and Tukey's HSD all-pairwise comparison test at 72 hours. Gene expression of TNFα for fish receiving 10 mcg of I2M2 was significantly greater (p=0.014) than fish receiving 0 mcg of I2M2 at 72 hours post-treatment.

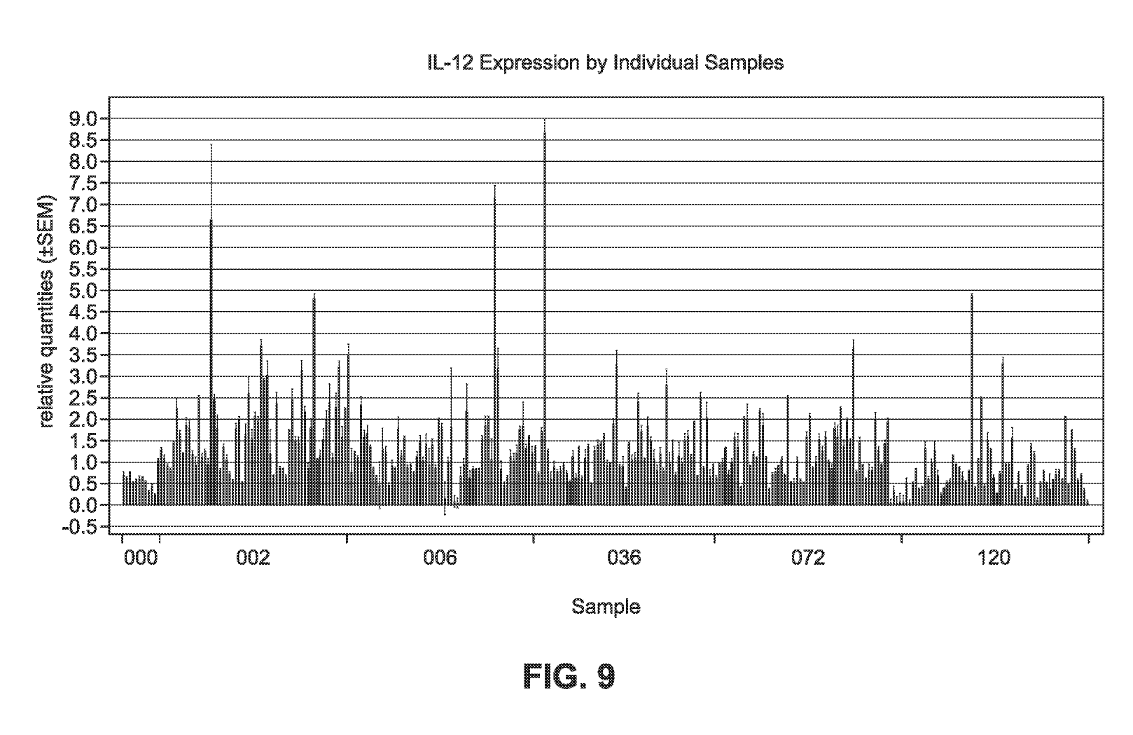

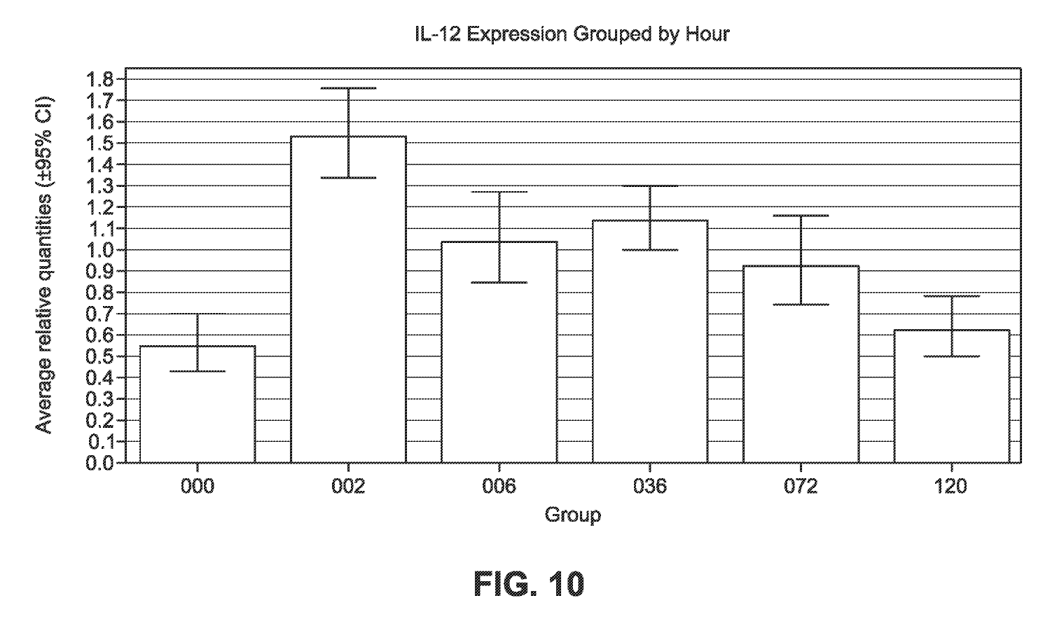

FIGS. 9 and 10 show the relative amount of IL-12 gene expression. A significant increase in IL-12 gene expression was found in the samples labeled “2 hours” with respect to basal samples (0 hours). Gene expression of IL-12 did not differ among treatment doses.

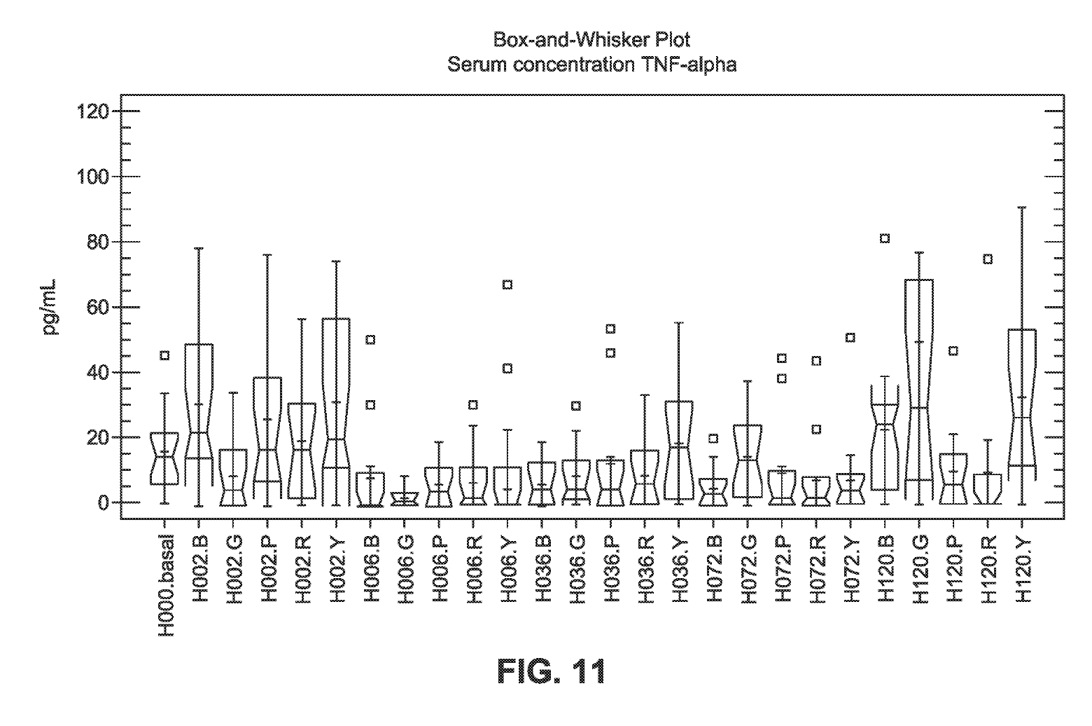

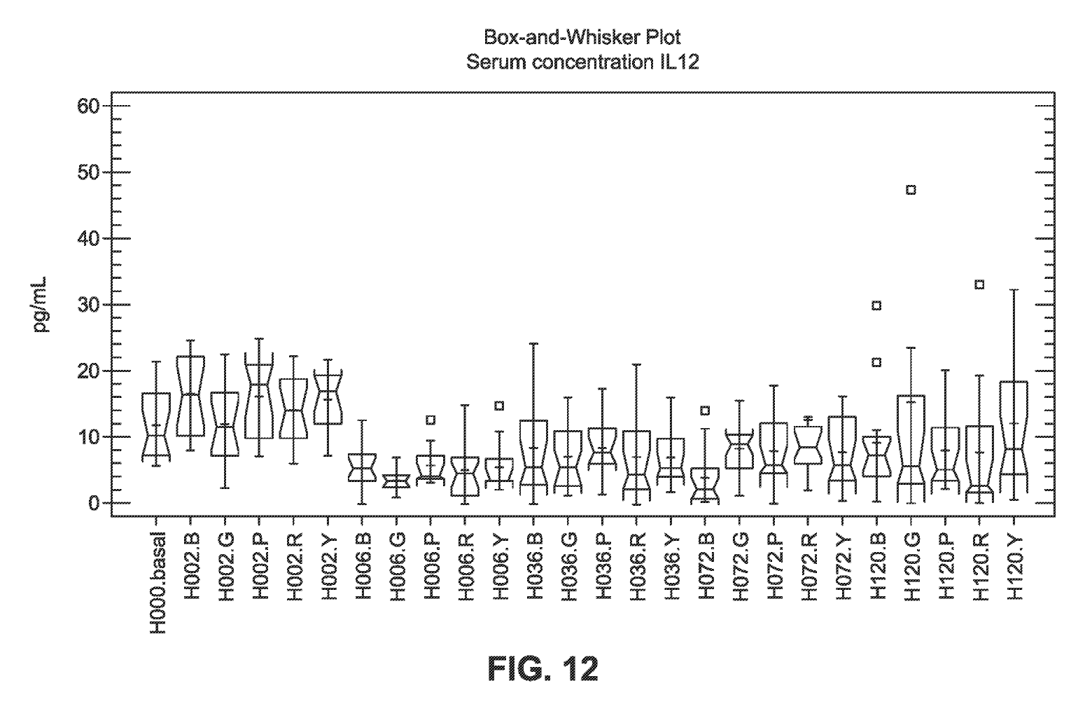

FIGS. 11 and 12 illustrate serum concentrations of TNFα and IL-12 respectively. A significant difference of both cytokine concentrations in serum was found between the group “2 hours” and the following groups. Serum concentrations of TNFα and IL-12 did not differ significantly among treatment doses.

It is contemplated that the I2M2 complex can be delivered to aquatic animals according to a variety of mechanisms, such as by injection or via introduction through the gastro intestinal system. In implementations, the I2M2 complex is administered to aquatic animals by injection across an exterior membrane, skin surface, and so forth of the animal. For example, in aquatic animals, the I2M2 complex can be administered via intraperitoneal injection.

### EXAMPLE 1

### Example Challenge Study

A challenge study utilizing the I2M2 complex was performed with Atlantic Salmon. As provided below, the challenge study indicated that specimens pre-treated with the I2M2 complex prior to challenging with Salmon Rickettsia Syndrome (SRS) showed significantly higher amounts of TNFα expression as compared to specimens not pre-treated with the I2M2 complex.

Acclimation—The specimens (also referred to as fish) included approximately 400 Atlantic Salmon (Salmo solar) of approximately 120 g average weight and were stocked in ten tanks of 350 L. In each tank were 40 specimens, with an average stock density of 10-15 kg/m³. The specimens acclimated to the tanks for a period of two weeks. During this period the fish were manually fed with EWOS Transfer 100 diet, where environmental parameters such as temperature and oxygen saturation were maintained at 12.3±1.2° C. and 103.1±4.1% respectively. During the acclimation period, each specimen was intraperitoneally tagged with a Passive Integrated Transponder (PIT) tag to identify each fish and associate it with a group. The treatment groups are provided below, with reference to Table 1.

PIT Tagging Procedure—The specimens were anesthetized with a dose of 17-20 mL of an anesthetic (AQUI-S™) in 100 L of water to induce deep sedation. The PIT tagging was facilitated by a sterile implanter. Prior to implantation a needle evaluation was made to avoid damage to specimen organs. The PIT tag was placed intraperitoneally in the belly, midline in front of pelvic fin.

Treatment Groups—The following table provides the assignment of treatments to the specimens:

TABLE 1
Treatment Schedule

| Treatment | | | Day 0 | Day 3 | | Day 17 | |
|---|---|---|---|---|---|---|---|
| Code | Tank ID | N | (Treatment) | (Challenge) | Day 14 | (Treatment) | Day 35 |
| T0 | C1 | 40 | Site Control | No Activity | 50% Euthanize | — | 50% Euthanize |
| T1 | C2 | 40 | Media | Media | 50% Euthanize | — | 50% Euthanize |
| T2 | R1, R2 | 20 | Media | SRS | 100% | — | — |
| | | | | | Euthanize | | |
| T3 | R1, R2 | 20 | 10 mcg | SRS | 100% | — | — |
| | | | I2M2 | | Euthanize | | |
| | | | Complex | | | | |
| T2 | R3, R4 | 20 | Media | SRS | — | — | Euthanize |
| | | | | | | | Remaining |
| T3 | R3, R4 | 20 | 10 mcg | SRS | — | — | Euthanize |
| | | | I2M2 | | | | Remaining |
| | | | Complex | | | | |
| T4 | C3 | 40 | — | Media | — | Media | Euthanize |
| | | | | | | | Remaining |
| T5 | R5, R6, | 20 | — | SRS | — | Media | Euthanize |
| | R7 | | | | | | Remaining |
| T6 | R5, R6, | 20 | — | SRS | — | 10 mcg | Euthanize |
| | R7 | | | | | I2M2 | Remaining |
| | | | | | | Complex | |

Treatment—On Day 0 (zero), groups T1, T2 and T3 received a preventive treatment (i.e., prior to exposure to a challenge material), in which groups T1 and T2 were treated with 0.1 mL of phosphate buffered saline (PBS) media solution (controls) and group T3 was treated with 0.1 mL of the I2M2 complex (100 micrograms/mL). Groups T4, T5, and T6 received a therapy treatment (i.e., following exposure to a challenge material), in which groups T4 and T5 were treated with a PBS media solution and group T6 was treated with 0.1 mL of the I2M2 complex (100 micrograms/mL). Treatments were performed with tuberculin syringes (1 ml) using the same procedure as the PIT tagging while avoiding the PIT tag injection point and organs.

Lethal Dose Determination—The determination of lethal dose 50 (LD50) of SRS was done with 198 fish from the same strain of the main trial. The fish were cultivated in six tanks of 180 L. The strain of Piscirickettsia salmonis was isolated with a titre of 10^{6.5} TCID₅₀/mL. Six groups of thirty-three (33) fish were injected with 0.2 mL of decreasing doses of inoculates: 1×10⁻¹, 1×10⁻², 1×10⁻³, 1×10⁻⁴, 1×10⁻⁵. A control group of thirty-three (33) fish was injected with saline solution (NaCl 0.9%). At the end of the LD50 trial, a dilution of 1×10⁻⁴ (approximately a titre of 10^{2.2} TCID50/mL) had been determined as the LD50.

SRS Challenge—the main SRS challenge was administered on Day 3, where groups T1, T2, T3, T4, T5, and T6 were challenged. In particular, Groups T1 and T4 were challenged with 0.2 ml of PBS as a negative control. Groups T2, T3, T5, and T6 were challenged with 0.2 ml of Piscirickettsia salmonis inoculated intraperitoneally. During the challenge period (e.g., lasting thirty-one (31) days), the specimens were manually fed with EWOS Transfer 100 diet and temperature and oxygen saturation environmental parameters were maintained at 15.0±1.7° C. and 101.7±9.4%, respectively.

Sampling—Samples were taken at two different points during the trial: Day 14 and Day 35. On Day 14, groups T0, T1, T2, and T3 were sampled. For negative controls (T0 and T1) twenty fish each were sampled (twenty for tank C1 and twenty for tank C2). For groups T2 and T3, the total fish in tanks R1 and R2 were sampled (seventy-nine (79) fish total). On Day 35, the total number of remaining fish was sampled (twenty fish of T0, nineteen fish of T1, eighteen fish of T2, fourteen fish of T3, forty fish of T4, twenty-nine fish of T5 and eighteen fish of T6).

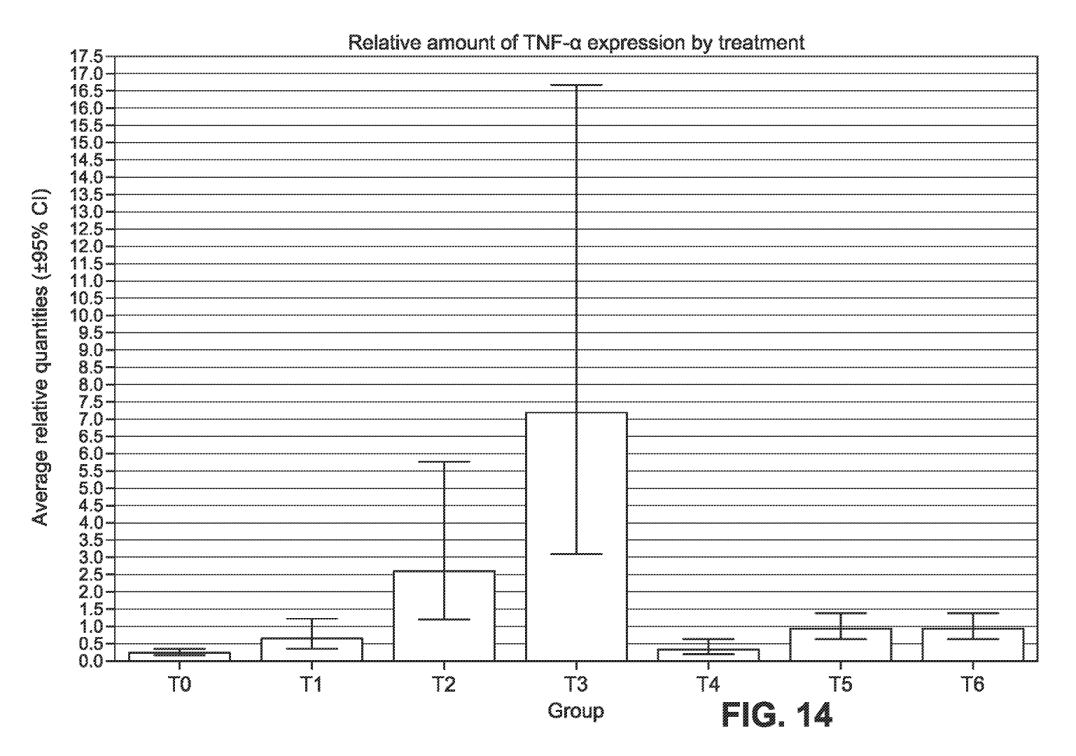

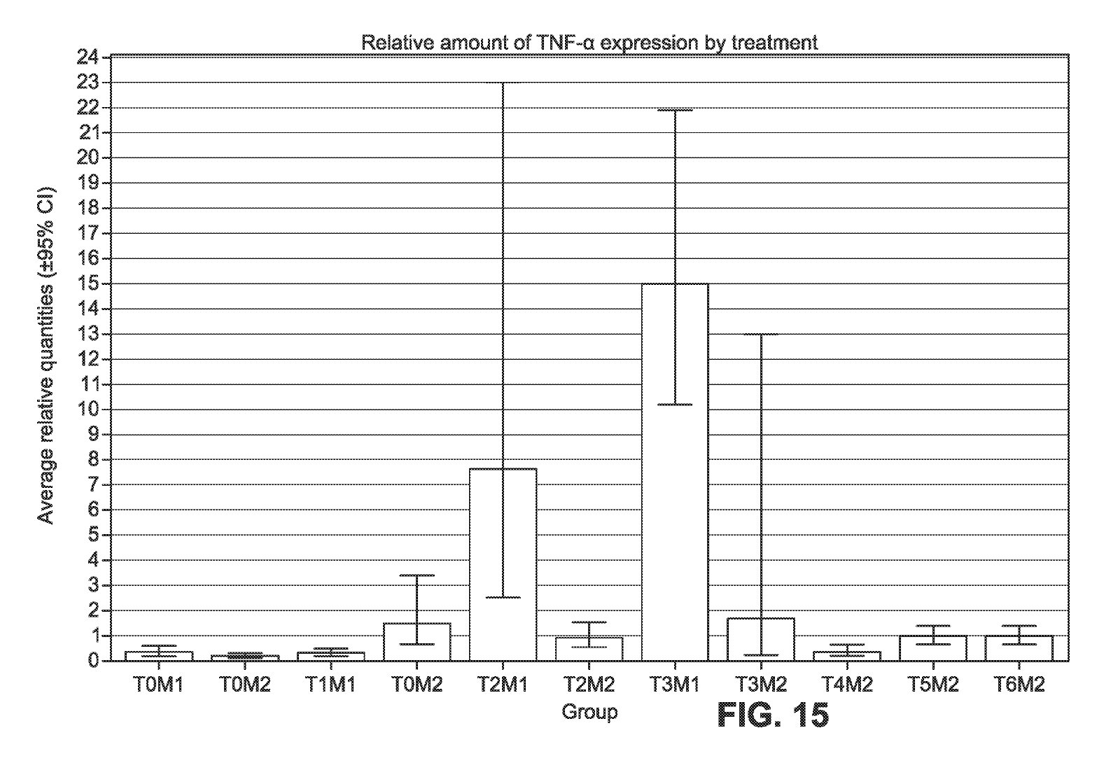

Results—The relative amount of TNFα expression shown by the specimens is provided in FIGS. 13 through 15. FIG. 13 shows a chart of TNFα expression by sample date resulting from the challenge study. As shown, M1 corresponds to the group of samples that were sampled on Day 14 of the challenge study, namely, groups T0, T1, T2, and T3, where for negative controls (T0 and T1) twenty fish each were sampled (twenty for tank C1 and twenty for tank C2), and for groups T2 and T3, the total fish in tanks R1 and R2 were sampled (seventy-nine fish total). M2 corresponds to the group of samples that were sampled on Day 35 of the challenge study, namely, the total number of remaining fish was sampled (twenty fish of T0, nineteen fish of T1, eighteen fish of T2, fourteen fish of T3, forty fish of T4, twenty-nine fish of T5 and eighteen fish of T6). FIG. 14 shows a chart of TNFα expression by treatment group resulting from the challenge study. As shown, the treatment group receiving the I2M2 complex on a preventative basis (e.g., T3—receiving the I2M2 complex on Day 0) displayed significant relative amount of TNFα expression, particularly in comparison with other treatment groups. FIG. 15 shows a chart of TNFα expression by treatment group and sample date resulting from the challenge study. As shown, M1 corresponds to the group of samples that were sampled on Day 14 of the challenge study, whereas M2 corresponds to the group of samples that were sampled on Day 35 of the challenge study. The group labeled as T3M1 corresponds to the treatment group T3 (received I2M2 complex on Day 0, challenged with SRS on Day 3) sampled on Day 14. As shown, the relative amount of TNFα expression for group T3M1 can suggest that when used preventatively, the I2M2 complex can provide a number of benefits, including but not limited to, proliferation of macrophages, increase cytokine expression, and increase activity of macrophages (e.g., for the uptake of bacteria). The results of the challenge study support the findings of the in vitro analysis described herein with regard to FIGS. 2-12, such that the I2M2 complex has shown to provide proliferation of macrophages, increase cytokine expression, and increase activity of macrophages. The translation of results from a laboratory-based in vitro study were corroborated by the study of the I2M2 complex with live fish, which can indicate an unexpected result of synergistic effects of the components of the I2M2 complex on live aquatic animals.

### CONCLUSION

Although the subject matter has been described in language specific to structural features and/or process operations, it is to be understood that the subject matter defined in the appended claims is not necessarily limited to the specific features or acts described above. Rather, the specific features and acts described above are disclosed as example forms of implementing the claims.

What is claimed is:

1. An innate immune modulating molecule complex, comprising:
a supercoiled covalently closed recombinant plasmid DNA, where the supercoiled covalently closed recombinant plasmid DNA comprises characteristics configured for inducing an innate immune response in aquatic animals; and
an adjuvant complexed with the supercoiled covalently closed recombinant plasmid DNA, where the adjuvant comprises characteristics configured for inducing cytokine release from macrophages in aquatic animals.

2. The innate immune modulating molecule complex as recited in claim 1, wherein the adjuvant is Vaxliant ENABL adjuvant.

3. The innate immune modulating molecule complex as recited in claim 1, wherein the supercoiled covalently closed recombinant plasmid DNA includes supercoiled circular covalently closed DNA strands (CCCD).

4. The innate immune modulating molecule complex as recited in claim 1, wherein at least a portion of the supercoiled covalently closed recombinant plasmid DNA is configured to bind to at least one of a Toll receptor on a macrophage endosomal organelle and a macrophage cytosolic receptor.

5. The innate immune modulating molecule complex as recited in claim 1, wherein the cytokine includes tumor necrosis factor alpha (TNFα).

6. The innate immune modulating molecule complex as recited in claim 1, wherein the aquatic animals include salmon.

7. A method, comprising:
selecting a supercoiled covalently closed recombinant plasmid DNA, where the supercoiled covalently closed recombinant plasmid DNA comprises characteristics configured for inducing an innate immune response in aquatic animals;
selecting an adjuvant, where the adjuvant comprises characteristics configured for inducing cytokine release from macrophages in aquatic animals; and
combining the supercoiled covalently closed recombinant plasmid DNA and the adjuvant to form a complex.

8. The method as recited in claim 7, wherein the adjuvant is Vaxliant ENABL adjuvant.

9. The method as recited in claim 7, further comprising:
introducing the complex to an aquatic animal.

10. The method as recited in claim 9, wherein introducing the complex to an aquatic animal includes:
introducing the complex to the aquatic animal as a preventative treatment.

11. The method as recited in claim 7, wherein at least a portion of the supercoiled covalently closed recombinant plasmid DNA is configured to bind to at least one of a Toll receptor on a macrophage endosomal organelle and a macrophage cytosolic receptor.

12. The method as recited in claim 7, wherein the supercoiled covalently closed recombinant plasmid DNA is derived via isolation as a recombinant DNA modified from bacteria plasmid.

13. The method as recited in claim 7, wherein the cytokine includes tumor necrosis factor alpha (TNFα).

14. A method, comprising:
introducing an innate immune modulating molecule complex to an aquatic animal, the innate immune modulating molecule complex including:
a supercoiled covalently closed recombinant plasmid DNA, where the supercoiled covalently closed recombinant plasmid DNA comprises characteristics configured for inducing an innate immune response in the aquatic animal; and
an adjuvant complexed with the supercoiled covalently closed recombinant plasmid DNA, where the adjuvant comprises characteristics configured for inducing cytokine release from macrophages in the aquatic animal.

15. The method as recited in claim 14, wherein the adjuvant is Vaxliant ENABL adjuvant.

16. The method as recited in claim 14, wherein the supercoiled covalently closed recombinant plasmid DNA includes supercoiled circular covalently closed DNA strands (CCCD).

17. The method as recited in claim 14, wherein at least a portion of the supercoiled covalently closed recombinant plasmid DNA is configured to bind to at least one of a Toll receptor on a macrophage endosomal organelle and a macrophage cytosolic receptor.

18. The method as recited in claim 14, wherein the cytokine includes tumor necrosis factor alpha (TNF-α).

19. The method as recited in claim 14, wherein the aquatic animal includes salmon.

20. The method as recited in claim 14, wherein introducing an innate immune modulating molecule complex to an aquatic animal includes:
introducing the innate immune modulating molecule complex to the aquatic animal as a preventative treatment.
